(19) United States
(12) Patent Application Publication
(10) Pub. No.: US 2016/0251289 A1
(43) Pub. Date: Sep. 1, 2016

(54) USE OF ALKANE SULFONIC ACID FOR PREPARING PHENOLIC ALCOHOL

(71) Applicant: ARKEMA FRANCE, Colombes (FR)

(72) Inventors: Jean-Alex LAFFITTE, Pau (FR); Bernard MONGUILLON, Bayonne (FR)

(73) Assignee: Arkema France, Colombes (FR)

(21) Appl. No.: 15/028,502

(22) PCT Filed: Oct. 21, 2014
(86) PCT No.: PCT/FR2014/052674

§ 371 Date: Apr. 11, 2016

(30) Foreign Application Priority Data
Oct. 22, 2013 (FR) 13.60268

(51) Int. Cl. C07C 37/08; C07C 45/53

(52) U.S. Cl. CPC C07C 37/08; C07C 45/53

(57) ABSTRACT

The use of alkane sulphonic acid for preparing phenolic alcohol. The invention relates to a use of at least one alkane sulphonic acid for preparing phenolic alcohol by decomposing aryl hydroperoxide, and to a method for preparing phenolic alcohol comprising the following successive steps: bringing aryl hydroperoxide into reaction in the presence of at least one alkane sulphonic acid, neutralising the reaction medium, separating the ketone or the aldehyde, separating phenolic alcohol by distillation.

The present invention relates to the use of an alkanesulfonic acid for the preparation of a phenolic alcohol. The invention also relates to a process for preparing a phenolic alcohol by decomposition of an aryl hydroperoxide.

The decomposition reaction of cumene hydroperoxide into phenol and acetone has been known for many years. This reaction corresponds to the following scheme:

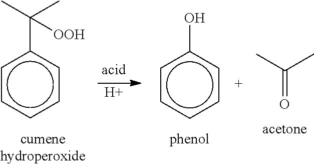

U.S. Pat. No. 2,757,209 and U.S. Pat. No. 2,737,527 dating from 1956 disclose this synthetic process. In general, cumene hydroperoxide, or the crude product from the hydroperoxidation reaction of cumene, is reacted with an acidic catalyst at a temperature of between 40 and 100° C. The reaction medium is then cooled and then neutralized with a base. The medium is then distilled so as to separate out first the acetone on a first distillation column, and then the heavier phenol on a second distillation column, which are the desired products. The acid used is generally a strong acid, generally in the form of a concentrated aqueous solution. The strong acids usually used are sulfuric acid, hydrochloric acid or phosphoric acid, which generate, respectively, after neutralization of the reaction medium with an aqueous base, sulfates, chlorides or phosphates.

Each of these acids has drawbacks, in terms of corrosion, or of generation of environmentally unfriendly effluents, to mention but a few of these drawbacks.

What is more, the presence of these salts (sulfates, chlorides and phosphates) in reality proves to be a great nuisance in several respects during the purification of phenol, most particularly during its subsequent distillation. Specifically, it was observed that sulfates, chlorides and phosphates are sparingly soluble in the acetone/phenol/water or phenol/water mixture. This observed low solubility may be harmful to the execution of the distillation and the recovery of the purified phenol under acceptable yield and purity conditions.

The presence of insoluble compounds in the streams of an industrial distillation installation is highly detrimental in the sense that these insoluble compounds may cause disruption of the streams, especially in the distillation column itself, and may consequently lead to pressure losses, or even risks of clogging, deposits, etc.

Furthermore, any pressure loss necessitates greater consumption of energy, and especially necessitates working at a higher temperature, the consequence of which is degradation and decomposition of the products, thus leading to a loss of quality of the purified phenol and to losses of overall distillation yield.

Improvements to this process have been proposed in the prior art.

The use of trichloromethanesulfonic acid leading to a smaller amount of acid used, to a decrease in the reaction temperature and to better yields is described in GB 803 480.

More recently, the use of an organic base to neutralize the reaction medium has been described in U.S. Pat. No. 6,201,157.

US 2003/0 088 129 describes a two-step process involving two different temperature stages.

The use of solid acidic catalysts, such as montmorillonite, cation-exchange resins or catalysts of alumina-silica type, has also been envisaged in JP 2007-099 746.

The inventors have found that the use of one or more alkanesulfonic acids leads to a marked improvement in the phenol preparation process.

Firstly, when compared with sulfuric acid, the alkanesulfonic acid is not dehydrating.

Specifically, in the case of phenol synthesis, it has been observed that the content of byproducts, especially of acetone, such as mesityl oxide, is smaller. As a result, a gain in selectivity and yield is observed.

Furthermore, neutralization of the reaction medium leads to the formation of insoluble salts, such as sodium or potassium sulfates, which need to be removed, generally by filtration, before performing the distillation. The use of an alkanesulfonic acid has the advantage of leading to the formation of organic salts, which are more soluble in the reaction medium. As a result, the filtration step becomes unnecessary. Furthermore, the risks of clogging due to the presence of salts are avoided. Consequently, in industrial terms, the use of an alkanesulfonic acid allows a gain in productivity by simplifying the process.

Thus, a first objective of the present invention is to use at least one alkanesulfonic acid for the preparation of a phenolic alcohol by decomposition of an aryl hydroperoxide, and preferably to use at least one alkanesulfonic acid for the preparation of a phenolic alcohol and a ketone or an aldehyde by decomposition of an aryl hydroperoxide.

Another objective consists in providing an improved process for preparing a phenolic alcohol, comprising a step of decomposition of an aryl hydroperoxide in the presence of at least one alkanesulfonic acid.

Yet other objectives will appear in the description of the present invention that follows.

Moreover, any range of values denoted by the expression “between a and b” represents the range of values extending from more than a to less than b (i.e. limits a and b excluded), whereas any range of values denoted by the expression “from a to b” means the range of values extending from a to b (i.e. including the limits a and b).

Use

The present invention relates to the use of at least one alkanesulfonic acid for the preparation of a phenolic alcohol by decomposition of an aryl hydroperoxide.

Aryl Hydroperoxide

For the purposes of the present invention, the term “aryl hydroperoxides” means compounds bearing aromatic groups, the hydroperoxide function of which is borne by a carbon atom positioned alpha to the aromatic ring.

The term “aromatic” refers to aromatic rings comprising from 6 to 14 carbon atoms, preferably from 6 to 10 carbon atoms (limits inclusive) and typically phenyl and naphthyl.

The aryl hydroperoxides according to the present invention are preferably of the general formula (or structure) (I) below:

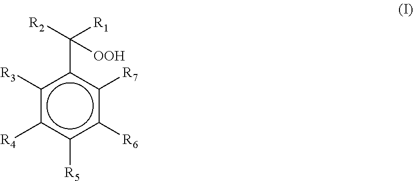

in which

the groups R₁ and R₂ denote, independently of each other, a hydrogen atom, a C₁ to C₁₈, preferably C₁ to C₁₀, more preferably C₁ to C₆ linear or branched alkyl radical, or a C₆ to C₁₄, preferably C₆ to C₁₀ aryl radical, and typically phenyl and naphthyl,

the groups R₃ to R₇ denote, independently of each other, a hydrogen atom, a C₁ to C₁₈, preferably C₁ to C₁₀, more preferably C₁ to C₆ linear or branched alkyl radical, a halogen atom, especially fluorine, chlorine, bromine and iodine, an —NO₂ radical, a —CN radical, an alkyl radical substituted with one or more halogen atoms, a C₆ to C₁₂ aryl radical, two adjacent groups R₃ to R₇ may together form one or more aliphatic or aromatic rings.

For example, R₃ and R₄ may together form an aromatic ring containing 6 carbon atoms, then leading to a naphthalene hydroperoxide. R₃ and R₄ may also together form an aromatic bicycle containing 12 carbon atoms, then leading to an anthracene hydroperoxide.

Preferably, the groups R₁ and R₂ are chosen from a hydrogen atom, a methyl, an ethyl, an n-propyl, an isopropyl, an n-butyl, an isobutyl and a tert-butyl.

Preferably, the groups R₃ to R₇ are chosen from a hydrogen atom, a methyl, an ethyl, an n-propyl, an isopropyl, an n-butyl, an isobutyl, a tert-butyl, a chlorine atom, a fluorine atom, a bromine atom and a phenyl radical.

According to a preferred embodiment, the group R₁ in the compound of formula (I) does not represent a hydrogen atom.

According to another preferred embodiment, the group R₂ in the compound of formula (I) does not represent a hydrogen atom.

According to yet another preferred embodiment, the groups R₁ and R₂ in the compound of formula (I) do not denote a hydrogen atom.

Preferably, the aryl hydroperoxide is chosen from cumyl hydroperoxide, butylbenzene hydroperoxide, ethylisopropylbenzene hydroperoxide, (propyl)naphthalene hydroperoxide, diisopropylbenzene hydroperoxide, sec-butylbenzene hydroperoxide, para-ethyl-isopropylbenzene hydroperoxide and alpha-isopropylnaphthalene hydroperoxide, preferably cumyl hydroperoxide.

As a result, decomposition in acidic medium of this aryl hydroperoxide of formula (I) makes it possible to lead to the formation of a phenolic alcohol and of a ketone or an aldehyde depending on the nature of the groups R₁ and R₂ and according to the following reaction scheme:

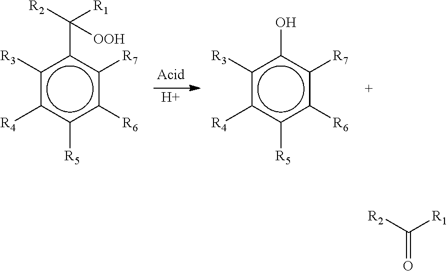

the groups R₁ to R₇ being identical to those described above.

Thus, for the purposes of the present invention, the term “phenolic alcohol” means phenol and phenols bearing substituents R₃ to R₇.

The compound of structure R₂C(═O)R₁ is referred to in the rest of the text as the co-product. It may be a ketone or an aldehyde depending on whether R₁ and/or R₂ represent(s) a hydrogen atom.

Preferably, cumyl hydroperoxide is used. Its decomposition in acidic medium makes it possible to lead to the formation of phenol and acetone.

Alkanesulfonic Acid

The decomposition of the aryl hydroperoxide into a phenolic alcohol is usually performed in acidic medium, or at the very least in the presence of acid(s), and preferentially in the presence of an aqueous solution of at least one alkanesulfonic acid.

In the present invention, the term “alkanesulfonic acid” means the acids of general formula (II) below:

R—SO₃H, (II)

in which the group R represents a saturated or unsaturated, linear or branched hydrocarbon-based chain comprising from 1 to 6 and preferably from 1 to 4 carbon atoms.

Preference is given to the compounds of formula (II) for which R represents a saturated, linear or branched hydrocarbon-based chain comprising from 1 to 6 and preferably from 1 to 4 carbon atoms.

The alkanesulfonic acids that may be used in the context of the present invention are most particularly preferably chosen from methanesulfonic acid, ethanesulfonic acid, n-propanesulfonic acid, isopropanesulfonic acid, n-butanesulfonic acid, isobutanesulfonic acid, sec-butanesulfonic acid, tert-butanesulfonic acid, and mixtures of two or more thereof in all proportions. The pKa values of the alkanesulfonic acids are all less than zero.

According to a most particularly preferred embodiment, the alkanesulfonic acid used in the context of the present invention is methanesulfonic acid or ethanesulfonic acid, and, most preferably, the acid used is methanesulfonic acid of formula CH₃SO₃H.

Any type of formulation comprising at least one alkanesulfonic acid may be suitable for use. It is possible to use at least one alkanesulfonic acid in anhydrous form or in aqueous solution form. As a general rule, the formulation comprises from 1% to 100% by weight of alkanesulfonic acid(s), preferentially from 1% to 99% by weight, more preferentially from 1% to 95% by weight, in general from 5% to 95% by weight and more generally from 5% to 90% by weight, in particular from 10% to 80% by weight of alkanesulfonic acid, and more particularly from 15% to 75% by weight, the remainder to 100% generally being constituted of water. It goes without saying that when the formulation comprises 100% by weight of alkanesulfonic acid(s), this means that the alkanesulfonic acid(s) are used pure, and more precisely are used alone, without addition of other formulation components.

The formulation also comprises the optional presence of one or more additives that are well known to those skilled in the art and, as nonlimiting examples, chosen from solvents, hydrotropes or solubilizers, biocides, disinfectants, rheological agents, preserving agents, surfactants, organic or mineral acids (for example sulfuric, phosphoric, nitric, sulfamic, acetic, citric, formic, actic, glycolic, oxalic and the like), foaming agents, antifoams, antifreezes (for example ethylene glycol, propylene glycol, and the like), colorants, fragrances, anticorrosion additives, UV stabilizers and other additives known to those skilled in the art, alone or as a mixture of two or more thereof in all proportions.

The formulation is, for example, an aqueous formulation which may be prepared in the form of a concentrated mixture that is diluted by the final user. As a variant, the formulation may also be a ready-to-use formulation, i.e. it does not need to be diluted. Use may be made, for example, of methanesulfonic acid as an aqueous solution sold by the company Arkema, for example an aqueous solution of methanesulfonic acid at 70% by weight in water, or anhydrous methanesulfonic acid, abbreviated as AMSA.

According to a preferred embodiment, the present invention relates to the use, for the preparation of phenol by decomposition of cumyl hydroperoxide in the presence of methanesulfonic acid (MSA) in all possible concentrations, ranging from AMSA (anhydrous MSA) to concentrations of the order of 5% by weight of MSA in water, and especially aqueous solutions of MSA at 70% by weight in water, sold by the company Arkema.

Needless to say, it is possible to use a mixture of at least one alkanesulfonic acid of formula (II), as has just been defined, in combination with one or more acids (organic and/or mineral), in all proportions.

However, it is preferred to use an alkanesulfonic acid or a mixture of alkanesulfonic acids alone.

Process

The invention also relates to a process for preparing a phenolic alcohol, comprising a step of decomposition of an aryl hydroperoxide in the presence of an alkanesulfonic acid.

More particularly, the process according to the invention comprises the following steps:

reaction of an aryl hydroperoxide in the presence of at least one alkanesulfonic acid,

neutralization of the reaction medium,

separation by distillation of the co-product, i.e. the ketone or the aldehyde, and then

separation by distillation of the phenolic alcohol.

The reagent for the process according to the invention is the aryl hydroperoxide as described above and more particularly cumyl hydroperoxide.

It may be used pure. It may also be present in a crude peroxidation reaction product.

For example, for the case of cumyl hydroperoxide, the crude reaction product from the hydroperoxidation step may comprise benzene derivatives, such as cumene, dimethylphenylcarbinol, dicumyl, dicumyl peroxide or acetophenone.

The acidic catalyst comprises at least one alkanesulfonic acid as described above and advantageously methanesulfonic acid. It may be used pure or as an aqueous solution.

Preferably, the content of alkanesulfonic acid(s) is between 100 ppm and 50 000 ppm, more particularly between 200 and 8000 ppm relative to the aryl peroxide(s) introduced.

The amount of alkanesulfonic acid(s) introduced into the crude reaction mixture may thus vary as a function of the reagent present, namely the pure aryl hydroperoxide or the crude hydroperoxidation reaction product. A person skilled in the art will know how to adapt the amount of alkanesulfonic acid(s) to be added to the crude reaction mixture as a function also of the concentration of said acid(s). According to one embodiment, the acid is introduced either into the stream of aryl hydroperoxide, or into the stream of solvent, this solvent generally being added as diluent, since the reaction is highly exothermic. This reaction is generally, usually, performed in the liquid phase.

The reaction solvent may be any organic solvent or mixture of organic solvents, optionally with water (aqueous-organic solvents) known to those skilled in the art and adapted to this type of reaction, and in particular one or more polar, protic or aprotic, preferably polar and aprotic, organic solvents. Ketones, and in particular acetone (dimethyl ketone), are most particularly suitable for performing the process according to the present invention.

The reaction temperature is generally between 50° C. and 150° C. A person skilled in the art will know how to adapt the reaction temperature as a function of the reagents present, the content of catalyst and the concentration of the reagents in the medium.

Similarly, the reaction time will depend on the abovementioned parameters.

According to one embodiment, the aryl hydroperoxide, which is pure or in the crude reaction product, is reacted, optionally but preferably in a solvent medium, with at least one alkanesulfonic acid, as described previously.

The process then comprises a step of neutralizing the reaction medium.

Neutralization of the acidic phase is not without consequences on the nature of the medium that is intended to be distilled. Specifically, during this neutralization step, the acidic species are neutralized in the form of salts.

The inventors have discovered, surprisingly, that the various alkali metal and/or alkaline-earth metal salts, present in this phase thus neutralized and intended to be distilled, are more soluble in the phenol/ketone/water or phenol/water mixture when the neutralization is performed on a medium that is acidified beforehand using at least one alkanesulfonic acid, in particular methanesulfonic acid, whereas the same salts are less soluble when the acidification is performed with other acids, especially the strong mineral acids commonly used in the field, such as sulfuric, hydrochloric or phosphoric acid.

In addition, the abovementioned salts in the form of alkanesulfonates, preferably methanesulfonates, proved to be more soluble than the sulfates, chlorides and other phosphates, in the phenol/ketone (or aldehyde)/water or phenol/water mixture (once the ketone or the aldehyde has been distilled off).

This is all the more noteworthy since distillation operations are very sensitive to solid impurities present in the distillation installations and especially in the distillation boiler (or stock vessel), but also in the distillation columns. Now, the phenol/ketone/water (or phenol/water) concentration gradients and the temperatures vary along the distillation columns.

Acidification with at least one alkanesulfonic acid, and preferably with methanesulfonic acid, offers the advantage of better solubility of the salts, especially of the sodium and/or potassium salts present in the phenol/ketone/water (or phenol/water) mixture.

This advantage of the alkanesulfonic acid over the other strong acids commonly used thus makes it possible to avoid the formation of solid deposits that might cause disruption of the streams, especially in the distillation column itself, and consequently result in pressure losses, or even risks of clogging, deposits, etc.

In addition, this greater solubility of the alkanesulfonic acid salts in the phase comprising the phenol, and especially in the boiler, at the bottom of the column, makes it possible to continue the distillation operation to a more advanced extent, and thus to further improve the distillation yield. Another advantage associated with better solubility of the salts in the phenol is the reduction of the risk of clogging at the bottom of the column, where the phenol/water mixtures are most concentrated in phenol. The overall yield for the distillation is thus greatly improved.

The greater solubility of the salts in the medium to be distilled may also make it possible to envisage a substantial reduction in the number of theoretical plates of the column, and consequently the physical height of the column, and similarly to substantially reduce the amount of energy used for the total distillation of the phenolic alcohol.

Yet another advantage, associated with acidification with at least one alkanesulfonic acid of the crude reaction product containing the phenolic alcohol, lies in the fact that the solid deposits are less substantial and, consequently, the periods of stoppage for cleaning the distillation installations are more spaced out over time.

Yet another advantage is that the alkanesulfonates and more particularly the methanesulfonates are highly soluble in aqueous medium and are biodegradable. The installations are consequently easier to clean and, as a result, require much smaller volumes of water, and the cleaning effluents are more environmentally friendly.

This neutralization uses basic substances. Preferably, the bases used are mineral species that are inert toward the species present in the reaction medium. More particularly, the bases are chosen from alkali metal or alkaline-earth metal hydroxides, such as sodium or potassium hydroxide, and alkali metal or alkaline-earth metal carbonates. These bases are usually and advantageously used in aqueous solution. Preferably, sodium hydroxide is used.

In the cases where insoluble salts are present, a filtration step may be envisaged, although this does not represent a preferred variant of the process of the invention. These insoluble substances may especially originate from the crude hydroperoxidation reaction product.

Preferably, a decantation step is performed so as to separate the aqueous phase originating from the neutralization solution and the organic phase containing the species to be purified.

The phenolic alcohol present in the neutralized reaction medium is then separated from the residual water and from the co-product. This separation may be performed according to any method known to those skilled in the art, and preferably by distillation. During this distillation operation, the co-product is first distilled off, then the water, and finally the phenolic alcohol.

Distillation of the phenolic alcohol is generally performed under reduced pressure, generally under vacuum (for example at 280 mmHg, i.e. 37.33 kPa) with the boiler at 170° C. and distillation of the phenol at about 150° C.

This process is characterized in that it uses at least one alkanesulfonic acid and more preferably methanesulfonic acid. The use of this acid has the advantage of being less corrosive, biodegradable and environmentally friendly, and also has the advantage of dissolving the salts present in the reaction medium, making it possible to perform the final distillation of the phenolic alcohol under more economical conditions, as has been explained earlier in the description.

Preferably, the process according to the invention comprises the following steps:

reaction of cumyl hydroperoxide in the presence of at least one alkanesulfonic acid, preferably methanesulfonic acid,

neutralization of the reaction medium,

separation by distillation of the acetone, and then

separation by distillation of the phenol.

The examples that follow illustrate the present invention without, however, limiting its scope defined by the claims which follow.

### EXAMPLES

### Comparison of the Solubilities

25 g of test solvent are placed in a 50 mL three-necked flask equipped with a condenser, a thermometer and a magnetic stirrer. The solvent is brought to the desired temperature and the alkanesulfonic acid salt is then added portionwise until cloudiness appears, indicating saturation of the medium.

The solubility is calculated in the following manner:

% salt solubility=m/(m+M)×100

with m=mass of dissolved salt, M=mass of solvent

The results are given in the tables below.

1. Solubility of the Salts in a Phenol/Acetone/Water (50/50/5) Weight Mixture:

A first measurement is taken at 20° C. at atmospheric pressure, and a second measurement is then taken at 60° C. The point at 60° C. makes it possible to evaluate the solubility of the species in the boiler during the distillation of the acetone. 1.1 Potassium Salts

TABLE 1

Temperature

| | 20° C. | 60° C. |
|---|---|---|
| Solubility CH₃SO₃K (mass %) | 0.80% | 0.80% |
| Solubility K₂SO₄ (mass %) | <0.12% | <0.12% |

In this mixture, at 20° C. and at 60° C., potassium methanesulfonate is more than 6.7 times more soluble than potassium sulfate.

1.2 Sodium Salts

TABLE 2

Temperature

| | 20° C. | 60° C. |
|---|---|---|
| Solubility CH₃SO₃Na (mass %) | 0.10% | 0.24% |
| Solubility Na₂SO₄ (mass %) | <0.10% | <0.10% |

In this mixture, at 60° C., sodium methanesulfonate is more than 2.4 times more soluble than sodium sulfate.

2. Solubility of the Salts in a Phenol/Water (95/5) Weight Mixture:

A first measurement is taken at 55° C. at atmospheric pressure, and a second measurement is then taken at 100° C. The point at 100° C. makes it possible to evaluate the solubility of the species in the boiler during the distillation of the water.

2.1 Potassium Salts

TABLE 3

Temperature

| | 55° C. | 100° C. |
|---|---|---|
| Solubility CH₃SO₃K (mass %) | 1.90% | 2.40% |
| Solubility K₂SO₄ (mass %) | 0.13% | <0.30% |

In this mixture, at 20° C. and at 60° C., potassium methanesulfonate is, respectively, 15 and 8 times more soluble than potassium sulfate.

2.2 Sodium Salts

TABLE 4

Temperature

| | 55° C. | 100° C. |
|---|---|---|
| Solubility CH₃SO₃Na (mass %) | 0.40% | 0.40% |
| Solubility Na₂SO₄ (mass %) | <0.20% | <0.20% |

In this mixture, at 20° C. and at 60° C., sodium methanesulfonate is more than twice as soluble as sodium sulfate.

1. A method of preparing a phenolic alcohol and a ketone or an aldehyde by decomposition of an aryl hydroperoxide, comprising using at least one alkanesulfonic acid.

2. The method as claimed in claim 1, wherein the aryl hydroperoxide is of structure (I) below: 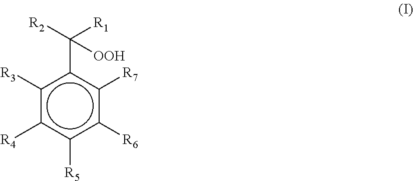
in which the groups R₁ and R₂ denote, independently of each other, a hydrogen atom, a C₁ to C₁₈ linear or branched alkyl radical, or a C₆ to C₁₄ aryl radical, the groups R₃ to R₇ denote, independently of each other, a hydrogen atom, a C₁ to C₁₈ linear or branched alkyl radical, a halogen atom, an —NO₂ radical, a —CN radical, an alkyl radical substituted with one or more halogen atoms, a C₆ to C₁₂ aryl radical, wherein two adjacent groups R₃ to R₇ may together form one or more aliphatic or aromatic rings.

3. The method as claimed in claim 2, wherein the groups R₁ and R₂ do not denote a hydrogen atom.

4. The method as claimed in claim 2, wherein the aryl hydroperoxide is cumyl hydroperoxide.

5. The method as claimed in claim 1, wherein the alkanesulfonic acid corresponds to the general formula (II) below:
R—SO₃H, (II)
in which the group R represents a saturated or unsaturated, linear or branched hydrocarbon-based chain comprising from 1 to 6 carbon atoms.

6. The method as claimed in claim 5, wherein the alkanesulfonic acid is methanesulfonic acid (CH₃SO₃H).

7. The method as claimed in claim 1, wherein the alkanesulfonic acid is in anhydrous form or in the form of an aqueous solution comprising from 5% to 90% by weight of sulfonic acid, the remainder to 100% being constituted of water.

8. A process for preparing a phenolic alcohol, comprising the following successive steps:
reacting an aryl hydroperoxide in the presence of at least one alkanesulfonic acid to obtain a reaction medium comprised of phenolic alcohol and ketone or aldehyde,
neutralizing the reaction medium,
separating the ketone or the aldehyde by distillation,
separating the phenolic alcohol by distillation.

9. The process as claimed in claim 8, in which the aryl hydroperoxide is cumyl hydroperoxide.

10. The process as claimed in claim 8, in which the alkanesulfonic acid is methanesulfonic acid.
